(19) United States
(12) Patent Application Publication
(10) Pub. No.: US 2010/0068799 A1
(43) Pub. Date: Mar. 18, 2010

(54) ORGANIC SUBSTANCE DETECTING DEVICE AND ITS MANUFACTURE METHOD

(75) Inventor: Michihiko Aki, Kawasaki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: FUJITSU LIMITED, Kawasaki-shi (JP)

(21) Appl. No.: 12/569,569

(22) Filed: Sep. 29, 2009

Related U.S. Application Data
(63) Continuation of application No. PCT/JP2007/000349, filed on Mar. 30, 2007.

(51) Int. Cl. C12M 1/34; B05D 5/12

(52) U.S. Cl. 435/287.2; 427/77

(57) ABSTRACT

A plurality of gold fine particles are discretely dispersed on and fixed to a principal surface of a substrate. Each of molecular probes is bonded to the gold fine particle at one end of the molecular probe. The molecular probe fixes a target capture portion at a tip thereof. The target capture portion has behavior of being specifically bonded to an organic molecule.

### CROSS-REFERENCE TO RELATED APPLICATION

This application is based upon and claims the benefit of priority of the prior International Application No. PCT/JP2007/000349, filed on Mar. 30, 2007, the entire contents of which are incorporated herein by reference.

### FIELD

The present invention relates to an organic substance detecting device and its manufacture method, utilizing molecular probes each having a target capture portion such as antibody at the tip thereof, the target capture portion specifically binding to a particular organic molecule such as a particular protein.

### BACKGROUND

Attention has been paid to an organic substance detecting device having a plurality of molecular probes coupled to a substrate, each molecular probe having antibody at the tip thereof, the antibody specifically binding to a particular protein. A conductive film serving as a working electrode of a two-electrode or three-electrode method is formed on the substrate surface, and a base of the molecular probe is coupled to the conductive film. Oligonucleotide molecule or the like is generally used for the molecular probe. Oligonucleotide molecule contains a number of negative charges of phosphoric acid so that it is charged negative.

As a positive potential is applied to the working electrode, a molecular probe lies on the substrate because of an electrostatic attractive force. Conversely, as a negative potential is applied to the working electrode, a molecular probe rises because of an electrostatic repulsive force. If protein is bound to the antibody at the tip, because of the inertia of protein, the posture of the molecular probe becomes hard to be changed. By detecting a change in the posture of a molecular probe, e.g., optically, it becomes possible to obtain information on a density of target proteins.

If molecular probes are distributed densely on a substrate, a free change in the posture of each molecular probe is disturbed by adjacent molecular probes. In order to allow a free change in the posture of each molecular probe, it is desired to properly control a distribution density of molecular probes.

Description will now be made on a method of controlling a distribution density of molecular probes and fixing the probes to a substrate.

A solution, in which molecular probes and alkanethiol are dissolved at a certain mole ratio, is formed. The base of each of the molecular probes is modified with a thiol group. A substrate having a gold surface is immersed in this solution. Since Au—S bonding reaction occurs, molecular probes and alkanethiol are bonded to the substrate surface.

FIG. 9A is a schematic diagram illustrating the state that molecular probes and alkanethiol are bonded to a substrate. An S atom at the base of a molecular probe 20 and an S atom at the base of alkanethiol 22 are bonded to the surface of a substrate 1. By adjusting a mole ratio of molecular probes and alkanethiol in the solution, it is possible to control a distribution density of molecular probes 20 to be bonded to the surface of the substrate 1 (e.g., the following Patent Document 1).

If molecular probes 20 form a colony on the substrate surface, a density of molecular probes 20 becomes locally high, and a change in the posture of the molecular probes 20 is disturbed. The alkanethiol 22 bonded to the surface of the substrate 1 has also a function of preventing the formation of a colony of molecular probes 20.

As illustrated in FIG. 9A, the molecular probe 20 includes a molecular wire 20b, and antibody 20c and fluorescent dye 20d fixed to the tip of the molecular wire 20b. The antibody 20c specifically binds to protein or the like serving as a particular target. The nucleotide chain 20c is charged negatively. As a negative potential is applied to the substrate 1, the molecular probe 20 rises up as illustrated in FIG. 9A because of an electrostatic repulsive force. Conversely, as a positive potential is applied to the substrate 1, the molecular probe 20 lies down as illustrated in FIG. 9B because of an electrostatic attractive force.

In the state that the molecular probe 20 lies down, even if excitation light is irradiated to the fluorescent dye 20d, emitted fluorescence is weak because of the quenching effects that the excitation energy moves partially to the substrate 1. By measuring an intensity of fluorescence, it is possible to estimate the posture of the molecular probe 20.

[Patent Document 1] Japanese Patent Laid-open Publication No. 2006-308373

### SUMMARY

With the method of immersing a substrate into solution to bond molecular probes and alkanethiol to the substrate surface, a distribution density of molecular probes 20 is adversely affected by convection of solution and diffusion of molecules which are difficult to be controlled artificially. Reproductivity of the distribution density is therefore insufficient.

A layer of alkanethiol 22 is involved between the fluorescent dye 20d and substrate 1 even in the state that the molecular probes 20 lie down on the substrate 1 as illustrated in FIG. 9B. Therefore, it is not possible for the fluorescent dye 20d to move sufficiently close to the surface of the substrate 1. It is not possible therefore to lower sufficiently the intensity of fluorescence.

An object of the present invention is to provide an organic substance detecting device and its manufacture method capable of improving reproductivity of a distribution density of molecular probes. It is another object of the present invention to provide an organic substance detecting device and its manufacture method capable of sufficiently lowering the intensity of fluorescence when molecular probes lie down on the substrate.

According to one aspect of the present invention, there is provided an organic substance detecting device comprising:

a plurality of gold fine particles discretely dispersed on and fixed to a principal surface of a substrate; and a plurality of molecular probes, each of which is bonded to the gold fine particle at one end of the molecular probe, and fixing a target capture portion at a tip of the molecular probe, the target capture portion having behavior of being specifically bonded to an organic molecule.

It is preferable to further fix fluorescent dye at the tip of the molecular probe.

According to another aspect of the present invention, there is provided a manufacture method for an organic substance detecting device comprising:

forming a working electrode made of conductive material different from gold over a support substrate;

discretely dispersing and fixing gold fine particles on a surface of the working electrode; and

fixing molecular probes to the gold fine particles at bases of the molecular probes, each of the molecular probes comprising a target capture portion at a tip thereof and a bonding portion at the base thereof, the target capture portion having a behavior of being specifically bonded to an organic molecule, and the bonding portion having a behavior of being bonded to gold.

Since molecular probes are bonded to gold fine particles, it is possible to control a distribution density of molecular probes by controlling a distribution density of gold fine particles. Since it is not necessary to bond alkanethiol or the like in an area where the molecular probes are not bonded, the tip of the molecular probe is closer to the substrate surface when the molecular probe lies down. Therefore, if fluorescent dye is fixed to the tip of the molecular probe, it is possible to further lower the intensity of fluorescence because of the quenching effects.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1A is a brief perspective view of a voltage driven type protein chip used by an organic substance detecting device of an embodiment, and FIG. 1B is a schematic diagram illustrating one gold particle and molecular probes bonded to the gold particle.

FIG. 2 is a schematic plan view illustrating one gold particle and sulfur atoms bonded to the gold particle.

FIG. 3 is a brief diagram illustrating a fine particle deposition system.

FIG. 4 is a brief diagram of a classification system to be used by the fine particle deposition system.

FIG. 5 is a brief diagram illustrating the whole structure of an organic substance detecting device of an embodiment.

FIG. 6A is a graph illustrating a time change in a potential of a working electrode, and FIG. 6B is a graph illustrating a time change in an intensity of fluorescence emitted from fluorescent dye.

FIGS. 7A and 7B are brief perspective views of a voltage driven type protein chip in a state of molecular probes rising up and in a state of molecular probes lying down, respectively, and FIG. 7C is a schematic diagram illustrating the molecular probes in a state of lying down.

FIG. 8 is a graph illustrating the actually measured results of a time change in fluorescence.

FIGS. 9A and 9B are schematic diagrams of a conventional voltage driven type protein chip in a state of molecular probes rising up and in a state of molecular probes lying down, respectively.

### DESCRIPTION OF EMBODIMENTS

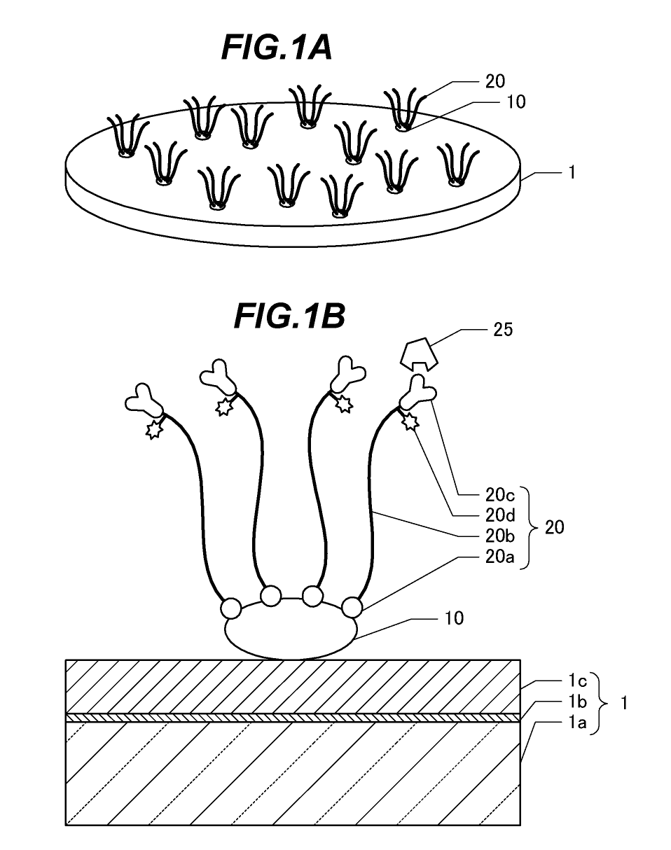

FIG. 1A is a brief perspective view of a voltage driven type protein chip used by an organic substance detecting device of the embodiment. Gold fine particles 10 are dispersively and separately fixed to a principal surface of a substrate 1 exposing material different from gold. A base of each molecular probe 20 is bonded to the gold fine particle 10. About four molecular probes 20 per gold fine particle are bonded.

FIG. 1B is a schematic diagram illustrating one gold fine particle 10 and molecular probes 20 bonded to the gold fine particle 10. The substrate 1 has the lamination structure that a working electrode 1c is formed on a base substrate 1a of sapphire or the like via a tight adhesion layer 1b. A thickness of the base substrate 1a is, e.g., about 350 μm. The tight adhesion layer 1b is made of, e.g., Ti and has a thickness of about 5 nm. The working electrode 1c is made of, e.g., Pt and has a thickness of about 40 nm. The gold fine particle 10 is fixed to the surface of the working electrode 1C. The size (assuming that the gold fine particle is a sphere, the size corresponds to a diameter) of the gold fine particle 10 is, e.g., about 0.9 nm.

The molecular probe 20 is constituted of a molecular wire 20b, a bonding portion 20a which is a basal portion of the molecular wire 20b, and a target capture portion 20c and fluorescent dye 20d fixed to the tip of the molecular wire 20b. A length of the molecular wire 20b is, e.g., about 15 nm. The bonding portion 20a contains an S (sulfur) atom. The molecular probe 20 is bonded to the gold fine particle 10 by Au—S bonding between the S atom and an Au atom of the gold fine particle 10.

The molecular wire 20b is made of, e.g., a nucleotide chain. A negative charge is distributed at a constant pitch in molecules of the nucleotide chain. Other ionic polymer may be used as the molecular wire 20b. Ionic polymers charged positively include polyguanidine. Ionic polymers charged negatively include polyphosphoric acid. The molecular wire 20b may be a single-chain, a double-chain, or a double-chain with a partial single-chain.

The target capture portion 20c captures an organic molecule (target organic molecule) 25 as a measurement subject by specifically binding to the organic molecule 25.

The target organic molecule 25 may be protein, blood plasma protein, tumor marker, apoprotein, virus, autoantibody, coagulation factor, fibrinolytic factor, hormone, drug in blood, nucleic acid, HLA antigen, lipoprotein, glycoprotein, polypeptide, lipid, polysaccharide, lipopolysacchride and the like.

The target capture portion 20c is constituted of antibody, antigen, enzyme, coenzyme or the like to a target. The target capture portion 20c may be a fragment of the antibody obtained through limited degradation of the antibody using protein degrading enzyme, or may be organic compound or biomacromolecule or the like having affinity with measuring target protein. As an example of the antibody, monoclonal immune globulin IgG antibody may be pointed to. As an example of the fragment derived from the IgG antibody, an Fab fragment of IgG antibody may be pointed to. A fragment derived from the Fab fragment may be used. As an example of organic compound having affinity with measuring target protein, enzyme matrix or its analog such as butanoic acid, pyruvic acid or tyrosine, coenzyme such as nicotinamide adenine dinucleotide (NAD), agonist such as diethylstilbestrol, brimonidine tartrate or 9-cis retinoic acid, antagonist such as tetrodotoxin, naloxone, 6-mercaptopurine or the like may be pointed to. If the above-described compound is unable to be directly bonded and fixed to the molecular wire 20b, the compound may be fixed by involving the bonding portion (generally a diatomic group) between the compound and the molecular wire 20b.

The fluorescent dye 20d emits florescence when it is excited by light energy. For example, as the fluorescent dye 20d, Fluorescein maleimide Cy3 (trade mark), or the like may be used.

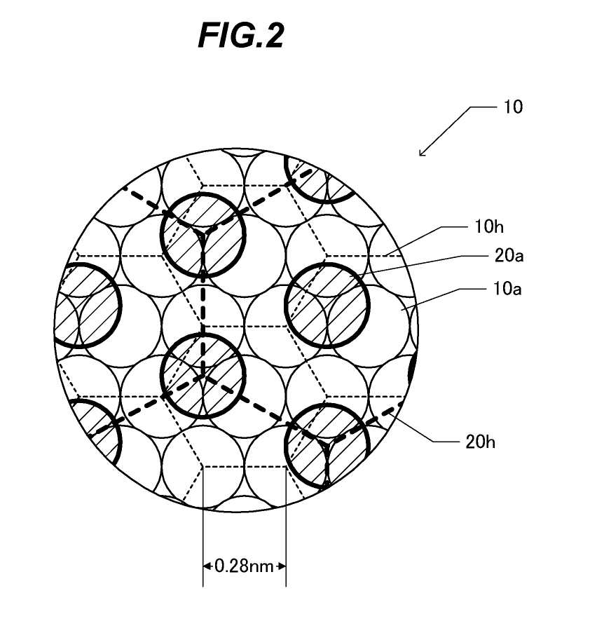

With reference to FIG. 2, description will be made on the number of molecular probes 20 capable of being bonded to one gold fine particle 10. It is assumed that the gold fine particle 10 is a sphere having a diameter of 0.9 nm. The surface area of the sphere facing toward the substrate is in contact with the substrate or is in close proximity to the substrate surface. Therefore, it is impossible for the molecular probe 20 to be bonded to the gold fine particle 10 in the surface area facing toward the substrate side, because of steric hindrance. An area where the molecular probe 20 is able to be bonded is substantially a half of the sphere surface. About twelve Au atoms are exposed in this area.

FIG. 2 illustrates a positional relation between Au atoms 10a constituting one gold fine particle 10 and S atoms 20a bonded to the gold fine particle 10. For the purposes of simplification, it is assumed that the surface of the gold fine particle 10 is a circle having a diameter of 0.9 nm and exposing the (111) plane. Au atoms 10a position at the lattice points of a hexagonal lattice 10h and the center of each hexagon, and a distance between the centers of adjacent Au atoms 10a is 0.29 nm. S atoms 20a position at points which are the centers of adjacent three Au atoms 10a and the lattice points of a hexagonal lattice 20h obtained by multiplying the hexagonal lattice 10h of the Au atoms by 3^{1/2} horizontally and vertically and rotating it by 30 degrees, and the center of each hexagon of the hexagonal lattice 20h.

Simply stated, about four S atoms are bonded to twelve Au atoms. Namely, about four molecular probes 20 are bonded to one gold fine particle 10.

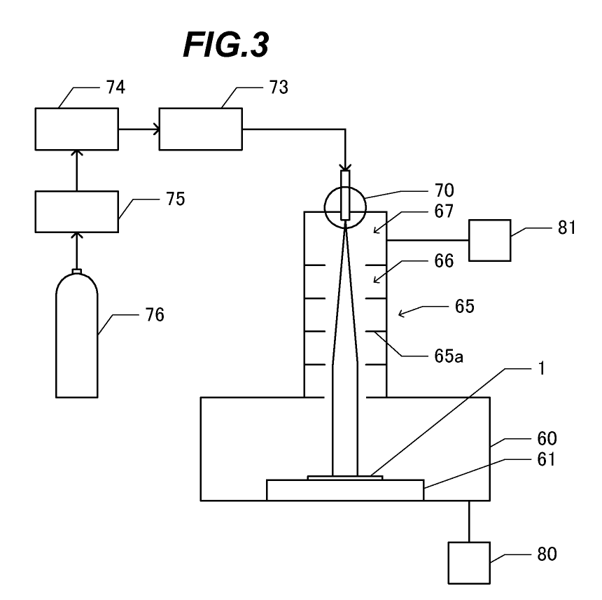

FIG. 3 is a brief diagram illustrating a fine particle deposition system for dispersing and fixing gold fine particles 10 to the surface of the substrate 1. This fine particle deposition system is disclosed, for example, in Japanese Patent Laid-open Publication No. 2006-117527.

A fine particle generator apparatus 75 generates gold fine particles by laser abrasion or evaporation condensation. For example, an Au target is placed in a fine particle generator chamber whose pressure is set to about 3×10³ Pa, and second harmonics of Nd:YAG laser at a repetition frequency of 20 Hz are entered onto the Au target to generate gold vapor. This vapor is cooled with carrier gas supplied from a carrier gas supply apparatus 76 to form gold fine particles by nucleus condensation. As the carrier gas, helium gas is used at a purity of 99.99995% and a flow rate of 1 SLM. Generated gold fine particles are transported by the carrier gas to a charging apparatus 74.

The charging apparatus 74 charges gold fine particles by radiation irradiation, ultraviolet irradiation or the like. For example, gold fine particles are heated in a tube type electric furnace to about 800° C., and charged by radiation from a radiation source of americium 241 (²⁴¹Am). Charged gold fine particles are transported to a classification apparatus 73. The classification apparatus 73 extracts gold fine particles having a desired size, by using a differential mobility analyzer (DMA) or the like.

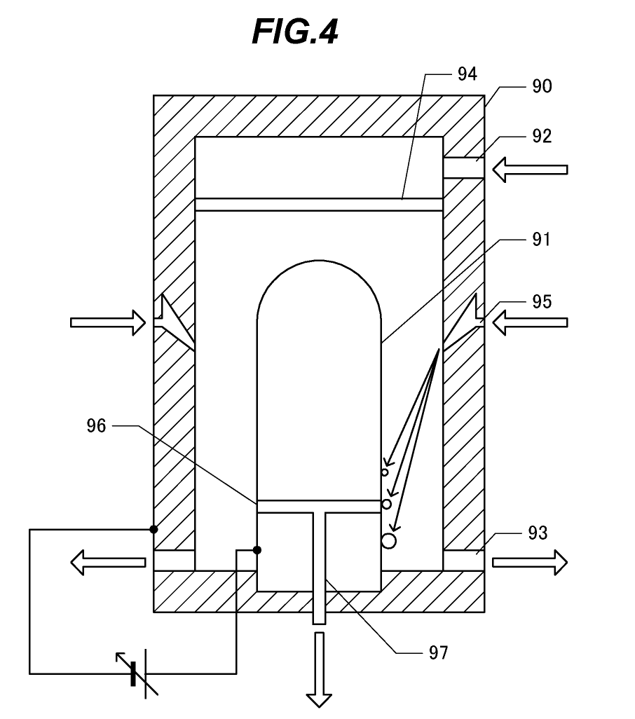

FIG. 4 is a brief diagram illustrating the classification apparatus 73. The classification apparatus 73 has a double tubular structure including an outside tube (outer tube) 90 and an inside tube (inner tube) 91. For example, an outer diameter of the inner tube 91 is 11 mm, and an inner diameter of the outer tube 90 is 18 mm. A dc voltage is applied across the outer tube 90 and inner tube 91. Sheath gas is introduced into the space between the outer tube 90 and inner tube 91 from a sheath gas inlet port 92 located near the upper end of the outer tube 90. The sheath gas passes through a filter 94 and is drained to the external from an outlet port 93 located at the lower end of the outer tube 90, via the space between the outer tube 90 and inner tube 91.

Upper slits 95 are formed on the outer tube 90, and lower slits 96 are formed on the inner tube 91. The lower slits 96 lie downstream of the upper slits 95 with respect to the sheath gas flow. A distance along an axial direction between the upper slits 95 and lower slits 96 is, e.g., 210 mm. Gold fine particles are introduced together with the carrier gas from the upper slits 95 into the space between the outer tube 90 and inner tube 91. The gold fine particles are attracted to the inner tube 91 by an electric field generated between the outer tube 90 and inner tube 91. An attractive velocity depends on the sizes of the gold fine particles. Therefore, only gold fine particles having certain sizes pass through the lower slits 96.

The gold fine particles passed through the lower slits 96 are transported to a nozzle 70 illustrated in FIG. 3 via a flow path 97 of the inner tube 91. By controlling a flow rate of the sheath gas and a voltage across the outer tube 90 and inner tube 91, gold fine particles having a desired size can be extracted (classified).

Reverting to FIG. 3, the carrier gas containing classified gold fine particles are introduced into a vacuum portion 67 of a deposition chamber 60 via the nozzle 70. The nozzle 70 has an orifice or a capillary. The inside of the deposition chamber 60 is differentially evacuated by vacuum pumps 80 and 81. The gold fine particles and carrier gas introduced into the vacuum portion 67 are transported to a high vacuum portion 66 made high vacuum by differential evacuation.

The gold fine particles transported to the high vacuum portion 66 is changed to a particle beam by the converging portion 65 including an electrostatic lens 65a. This particle beam is irradiated to the substrate 1 placed on a movable stage 61. The gold fine particles are therefore distributed on the substrate 1 almost uniformly and fixed thereto. A size of the gold fine particles obtained in the above-described method is distributed within a range of 10% either side of the average particle diameter. It is possible to control a surface density of gold fine particles deposited on the substrate 1 with good reproductivity, by changing a laser output and laser radiation time of the fine particle generator apparatus 75.

The gold fine particles 10 may be formed by other methods. For example, a gold film having a thickness in a range of 0 monoatomic layer thickness to 3 monoatomic layer thickness deposited on the substrate 1, and thereafter heat treatment is performed for about one hour at about 500° C. Because of a variation in a gold film thickness, islands, i.e., gold fine particles having a particle size of about 0.9 nm, are formed during heat treatment.

Next, description will be made on a method of bonding the molecular probes 20 to the gold fine particles 10. Molecular probes 20 are prepared. One end (basal portion) of each of the molecular probes 20 is modified by an alkanethiol group, e.g., mercaptohexanol (MCH) derivative, 5′-Thiol-Modifier C6 (trademark). The target capture portion 20c and fluorescent dye 20d are fixed at the tip of each of the molecular probes 20. The molecular probes 20 may be synthesized by chemical synthesis, fermentative production or the like. Commercially available molecular probes may also be used.

The molecular probes 20 are dispersed or dissolved in solvent. Solvent may be water, alcohol, liquid containing pH buffer and interfacial active agent or the like. The substrate 1 having gold fine particles 10 bonded to the surface thereof is immersed in this solution. S atoms of the thiol group are bonded to Au atoms of the gold fine particles 10 so that the molecular probes 20 are bonded to the gold fine particles 10. The molecular probes 20 are hard to be bonded to the working electrode 1c made of Pt, W, Ir or Rh.

A distribution density of molecular probes 20 is therefore determined by a distribution density of gold fine particles 10. Since it is possible to control the distribution density of gold fine particles 10, controllability of the distribution density of molecular probes 20 is improved.

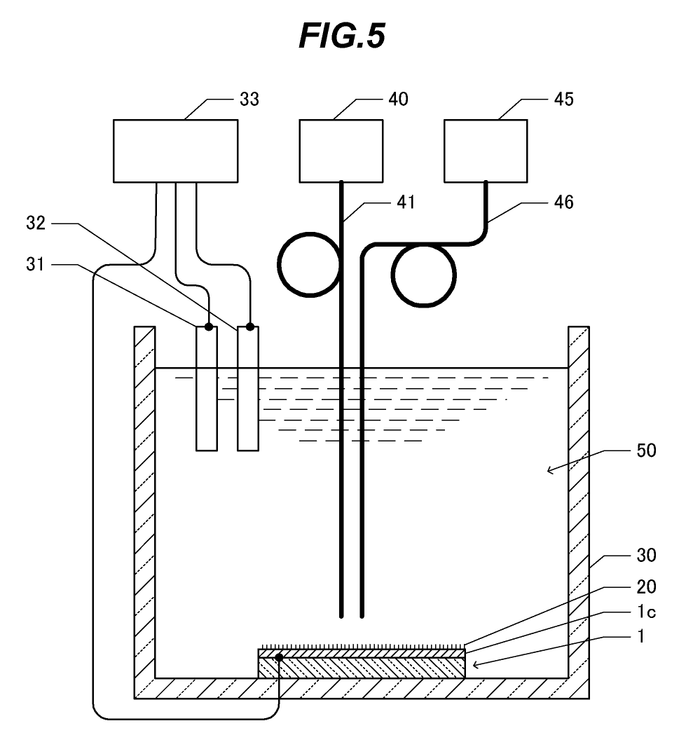

FIG. 5 is a brief diagram illustrating the whole structure of an organic substance detecting device. Sample solution 50 is accommodated in a container 30. The sample solution 50 is buffer solution in which measuring target protein or the like is dissolved, and is, for example, Tris-HCl 10 mM pH7.4, 50 mM NaCl. The voltage driven type protein chip illustrated in FIG. 1 is immersed in the sample solution 50.

Excitation light is emitted from an excitation light source 40. The emitted excitation light is irradiated to a fluorescent dye 20d of the voltage drive type protein chip via an optical fiber 41. The excitation light source 40 may be an Ar laser at an oscillation wavelength of 514.5 nm and an output of 500 μW.

Fluorescence generated by the fluorescent dye 20d is guided to a photo detector 45 via another optical fiber 46. If Cy3 (trademark) is used as the fluorescent dye 20d, a fluorescent spectrum has a spread in a wavelength range of 520 nm to 750 nm. The photo detector 45 measures an intensity of fluorescence at a particular wavelength, e.g., at a wavelength of 565 nm.

An opposite electrode 31 and a reference electrode 32 are immersed into the sample solution 50. The opposite electrode 31 is, for example, made of Pt. The reference electrode 32 is an electrode to be generally used in the three-electrode method, and is, for example, made of Al/AgCl (3M KCl). The working electrode 1c of the voltage driven type protein chip, the opposite electrode 31 and the reference electrode 32 are connected to a measurement power source 33.

The measurement power source 33 applies a voltage across the working electrode 1c and the opposite electrode 31 in such a manner that a potential of the working electrode 1c a predetermined level taking the reference electrode 32 as a reference point of potential.

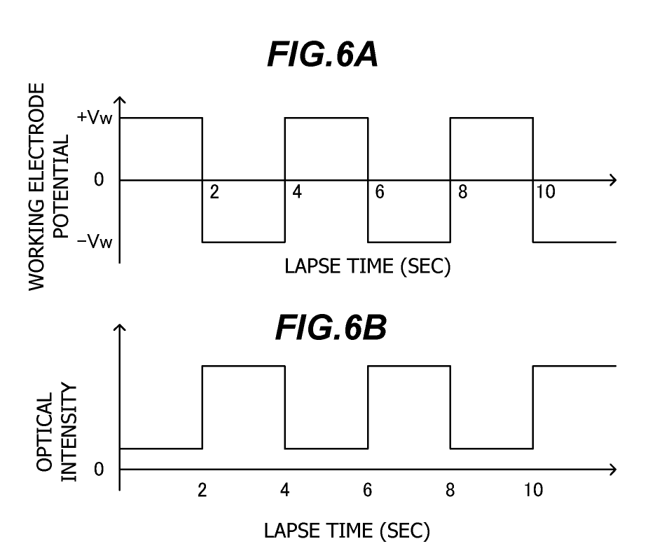

FIG. 6A illustrates an example of a time change in a potential of the working electrode 1c. The abscissa represents a lapse time in the unit of “second”, and the ordinate represents a potential of the working electrode 1c. An absolute value of a potential of the working electrode 1c is Vw, and its polarity reverses every 2 seconds. Namely, a change in a potential of the working electrode 1c demonstrates a rectangular wave form having a period of 4 seconds and a frequency of 0.25 Hz. An absolute value Vw of the potential is, for example, 200 mV.

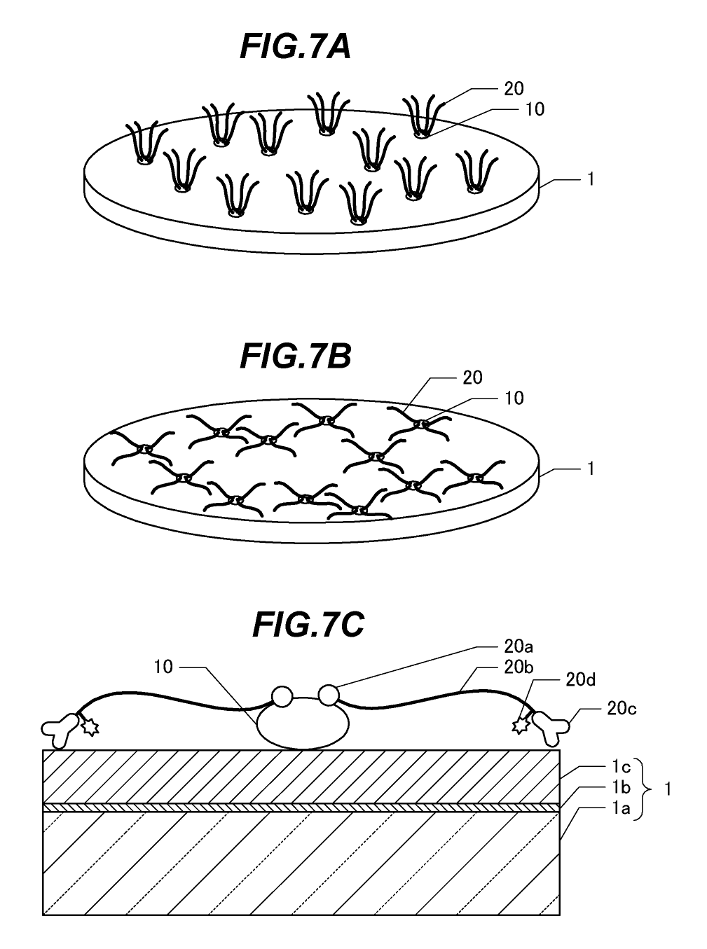

FIGS. 7A and 7B illustrate the states of molecular probes when a potential of the working electrode 1c negative and positive, respectively. The molecular wire 20b constituting the molecular probe 20 is charged negatively. Therefore, when a potential of the working electrode 1c negative, the molecular probe 20 rises up as illustrated in FIG. 7A, whereas when a potential of the working electrode 1c positive, the molecular probe 20 lies down as illustrated in FIG. 7B.

FIG. 6B illustrates a time change in an optical intensity measured with the photo detector 45. The abscissa represents a lapse time in the unit of “second”, and the ordinate represents an optical intensity. During a period while a potential of the working electrode 1c negative, the molecular probe 20 rises up so that the quenching effects are not developed because of the metal film of the working electrode 1c, and an optical intensity is relatively high. During a period while a potential of the working electrode 1c is positive, the molecular probe 20 lies down so that an optical intensity is relatively low because of the influence of the quenching effects.

If a frequency (hereinafter called “measurement frequency”) at which the polarity of a potential at the working electrode 1c reverses is as low as about 0.2 Hz, the molecular probe 20 changes its posture by following a change in the potential. However, as the measurement frequency is made high (e.g., 1 kHz), it is not possible for a change in the posture of the molecular probe 20 to follow a change in the potential. Therefore, amplitude of an optical intensity measured with the photo detector 45 lowers. In the state that the target capture portion 20c captures a target organic molecule 25, the upper limit of the frequency, at which a change in the posture of the molecular probe 20 being able to follow a change in the potential, lowers (frequency response degrades) because of the mass of the target organic molecule 25.

In accordance with a lowered amplitude of an optical intensity of fluorescence or a degraded frequency response of a change in the posture of the molecular probe 20, it is possible to measure a density of target organic molecules in the sample solution 50 with high sensitivity and rapidly.

Since a distance between a measuring target protein captured by the molecular probe 20 and the fluorescent dye is short (e.g., several to 100 nm), the captured protein absorbs (quenches) fluorescence. Namely, as the measuring target protein is captured by the molecular probe 20, an optical intensity measured with the photo sensor 45 lowers. By detecting this lowering of the optical intensity, it is also possible to measure a density of measuring target proteins.

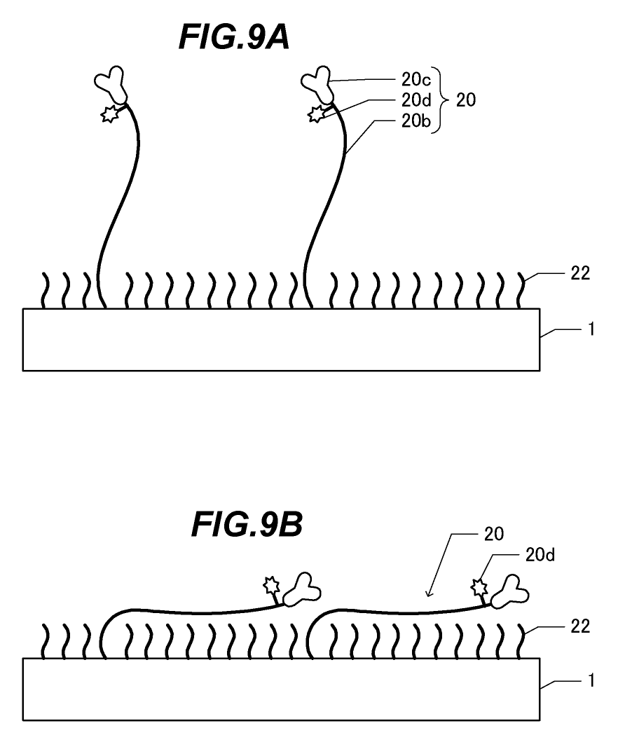

FIG. 7C schematically illustrates molecular probes lying down on a substrate. In a conventional example illustrated in FIG. 9B, the layer including alkanethiol 22 is involved between the fluorescent dye 20d and the surface of the substrate 1 (i.e., working electrode) in the state that the molecular probes 20 lie down on the substrate. In contrast, in the embodiment, the surface of the working electrode 1c is not covered with alkanethiol or the like, but is exposed. The fluorescent dye 20d is closer to the working electrode 1c. Therefore, the large quenching effects are demonstrated. An optical intensity during the period while a potential of the working electrode 1c is positive is therefore weaker. Namely, a difference between optical intensities is large during the two periods while the polarities at the working electrode 1c are different.

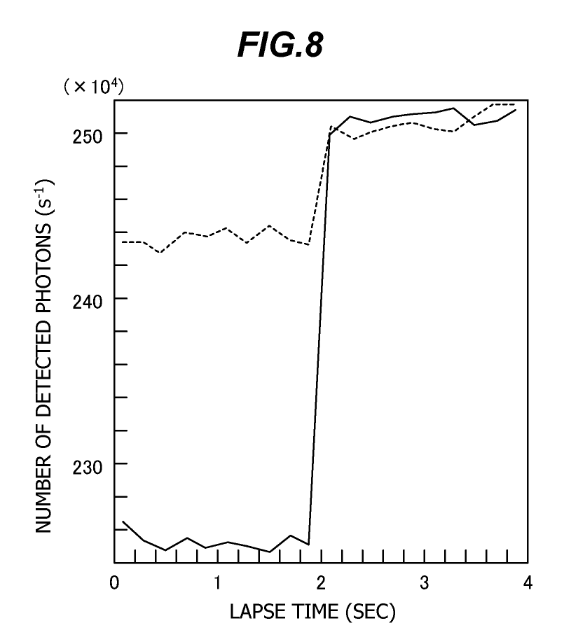

FIG. 8 illustrates an example of measurement results of an optical intensity. The abscissa represents a lapse time in the unit of “second”, and the ordinate represents the number of detected photons in the unit of “second⁻¹”. A solid line indicates an optical intensity measured by the organic substance detecting device of the embodiment, and a broken line indicates an optical intensity of comparative example using the protein chip illustrated in FIG. 9B. A unit time for counting the number of photons was set to 200 ms. The lapse time of the abscissa illustrated in FIG. 8 is therefore plotted at a unit time of 200 ms. The measurement results illustrated in FIG. 8 correspond to numerical integration values of measurement results during 15 minutes.

It is seen that amplitude of an optical intensity detected by the organic substance detecting device of the embodiment is larger than that measured by the organic substance detecting device of the comparative example. Particularly in the embodiment, an intensity of fluorescence lowers when a positive potential is applied to the working electrode 1c. Since amplitude of an optical intensity is large, it is possible to perform high reliability measurements at high sensitivity by eliminating the influence of noises.

In the above-described embodiment, although the molecular probe 20 having a length of about 15 nm is used, a length of the molecular probe 20 may be 2 to 100 nm. If the molecular probe 20 is too short, quenching by the working electrode is not released even if the molecular probe 20 rises so that amplitude of an optical intensity lowers. It is therefore difficult to increase detection sensitivity. Conversely, if the molecular probe 20 is too long, a distribution density of molecular probes 20 is required to be lowered, in order to allow the posture of the molecular probe 20 to change freely. If a distribution density of the molecular probes 20 is low, an intensity of fluorescence lowers. As a length of the molecular probe 20 exceeds 100 nm in particular, since the distribution density is required to be lowered, fluorescence intensity becomes the same level as the noise level of the photo detector 45 even in the state that the molecular probe 20 rises up.

In the above-described embodiment, although a diameter of the gold fine particle is set to about 0.9 nm, the diameter may be 0.45 to 1.2 nm. If the gold fine particle is too small, an S atom is unable to be stably bonded to the gold fine particle. In order to make at least one S atom be stably bonded, it is preferable that a diameter of the gold fine particle is equal to or longer than 4.5 nm. If the gold fine particle is too large, the number of S atoms bonded to one gold fine particle is large, and the molecular probes are locally concentrated. As the molecular probes are concentrated, change in the postures of the molecular probes is hard to occur. It is therefore preferable that a diameter of the gold fine particle is equal to or shorter than 1.2 nm. About seven molecular probes are bonded to a gold fine particle having a diameter of 1.2 nm.

If a distribution density of the molecular probes 20 on a substrate is too high, the molecular probe 20 contacts adjacent molecular probes 20 and is hard to lie down on the substrate. Conversely, if a distribution density of the molecular probes 20 is too low, detection sensitivity lowers. In order to allow the molecular probe 20 having a length of about 15 nm to easily lie down on the substrate, an upper limit of a preferable range of a distribution density of the molecular probes 20 on the substrate surface is about 1.4×10¹⁵ probes/m². Since about four molecular probes 20 are bonded to one gold fine particle 10, an upper limit of a preferable range of a distribution density of the gold fine particles 10 is about 3.5×10¹⁴ particles/m².

In order to retain a sufficient detection sensitivity, a distribution density of the molecular probes 20 is preferably set in such a manner that a fluorescence intensity when the molecular probe 20 rises up is about ten times as large as the noise level of the photo detector 45. For example, a distribution density of molecular probes 20 is preferably set to be equal to higher than 3×10¹³ probes/m². If a noise level of a fluorescence intensity measuring system is low, a distribution density of the molecular probes 20 may be lowered.

An upper limit Amax (particles/m²) of a preferable range of a distribution density of the gold fine particles 10 is calculated by an equation of:

Amax=3/(20 [symbol] ²L²r²×10⁻¹⁸)

where L (nm) represents a length of the molecular probes 20, and r (nm) represents a radius of the gold fine particles 10. The process of deriving this equation will be described below.

The maximum surface density Pmax (probes/m²) of the molecular probes 20 having a length of L and being able to lie down without mutual interference is able to be represented by:

Pmax=1/( [symbol] L²×10⁻¹⁸)

The number N (probes/particle) of the molecular probes 20 capable of being stably bonded to Au atoms exposed on a surface of the gold fine particle 10 having a radius r is given by:

N=20 [symbol] r²/3

It is herein assumed that an area excluded by an Au atom exposed on the gold fine particle is 0.1 nm², that one S atom per three Au atoms is bonded, and that an area where an S atom is able to be stably bonded without steric hindrance on the working electrode surface is a half of a sphere surface. The upper limit Amax (particles/m²) of a preferable range of a distribution density of gold fine particles 10 is calculated by an equation of:

Amax=Pmax/N=3/(20 [symbol] ²L²r²×10⁻¹⁸)

It is generally preferable to set a distribution density (particles/m²) of gold fine particles to be equal to or lower than:

3/(20 [symbol] ²L²r²×10⁻¹⁸)

where r (nm) represents an average of radii of gold fine particles and L (nm) represents an average of lengths of molecular probes 20.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present invention have been described in detail, it should be understood that the various changes, substitutions, and alterations could be made hereto without departing from the spirit and scope of the invention.

What is claimed is:

1. An organic substance detecting device comprising:
a plurality of gold fine particles discretely dispersed on and fixed to a principal surface of a substrate; and
a plurality of molecular probes, each of which is bonded to the gold fine particle at one end of the molecular probe, and fixing a target capture portion at a tip of the molecular probe, the target capture portion having behavior of being specifically bonded to an organic molecule.

2. The organic substance detecting device according to claim 1, wherein a diameter of the gold fine particle is in a range of 0.45 nm to 1.2 nm.

3. The organic substance detecting device according to claim 1, wherein a distribution density (particles/m²) of the gold fine particles is equal to or lower than 3/(20 [symbol] ²L²r²×10⁻¹⁸), where r (nm) represents an average of radii of the gold fine particles and L (nm) represents an average of lengths of the molecular probes.

4. The organic substance detecting device according to claim 1, wherein the molecular probes are bonded to the gold fine particles by Au—S bandings.

5. The organic substance detecting device according to claim 1, wherein the molecular probes contain nucleotide chains.

6. The organic substance detecting device according to claim 1, wherein the substrate comprises a working electrode formed over the principal surface and made of conductive material different from gold, and the gold fine particles are
fixed to the working electrode.

7. The organic substance detecting device according to claim 6, wherein fluorescent dye is further fixed to the tip of each of the molecular probes.

8. The organic substance detecting device according to claim 7, further comprising:
a container for accommodating the substrate and sample solution as a measuring target;
a light source for irradiating excitation light to the molecular probes fixed to the substrate;
a photo detector for detecting luminescence radiated from fluorescent dye fixed to the tip of each of the molecular probes;
an opposite electrode immersed in the sample solution accommodated in the container and paired with the working electrode formed over the substrate;
a reference electrode for applying a reference potential to the sample solution accommodated in the container; and
a power source for measuring a potential of the working electrode with respect to the reference electrode, and applying a voltage across the working electrode and the opposite electrode in such a manner that a polarity of the potential of the working electrode reverses periodically.

9. The organic substance detecting device according to claim 8, wherein the power source is able to change a period of reversing the polarity of the potential of the working electrode.

10. A manufacture method for an organic substance detecting device comprising:
forming a working electrode made of conductive material different from gold over a support substrate;
discretely dispersing and fixing gold fine particles on a surface of the working electrode; and
fixing molecular probes to the gold fine particles at bases of the molecular probes, each of the molecular probes comprising a target capture portion at a tip thereof and a bonding portion at the base thereof, the target capture portion having a behavior of being specifically bonded to an organic molecule, and the bonding portion having a behavior of being bonded to gold.

11. The manufacture method for an organic substance detecting device according to claim 10, wherein a diameter of each of the gold fine particles is in a range of 0.45 nm to 1.2 nm.

12. The manufacture method for an organic substance detecting device according to claim 11, wherein the gold fine particles are dispersed in such a manner that a distribution density (particles/m²) of the gold fine particles is equal to or lower than 3/(20 [symbol] ²L²r²×10⁻¹⁸), where r (nm) represents an average of radii of the gold fine particles and L (nm) represents an average of lengths of the molecular probes.

13. The manufacture method for an organic substance detecting device according to claim 10, wherein the base of each of the molecular probes has a thiol group or a dithiol group.

14. The manufacture method for an organic substance detecting device according to claim 10, wherein the molecular probe contains a nucleotide chain.

15. The manufacture method for an organic substance detecting device according to claim 14, wherein fluorescent dye is further fixed to the tip of each of the molecular probes.
